(19) United States
(12) Patent Application Publication
(10) Pub. No.: US 2015/0141630 A1
(43) Pub. Date: May 21, 2015

(54) PROCESS FOR PREPARATION OF IRON SUCROSE

(71) Applicant: Dr. Reddy's Laboratories Limited, Hyderabad (IN)

(72) Inventors: Amit Biswas, Hyderabad (IN); Vilas Dahanukar, Hyderabad (IN); Elati Ravi Ram Chandrasekar, Hyderabad (IN); Mohanarangam Saravanan, Valavanur (IN); Penumandla Raja Gopal, Warangal (IN); Chandra Sekhar Vempati, Vijayawada (IN); Kushal Surajmal Manudhane, Medak (IN); Ande Sameer Kumar, Hyderabad (IN)

(21) Appl. No.: 14/550,260

(22) Filed: Nov. 21, 2014

(30) Foreign Application Priority Data
Nov. 21, 2013 (IN) 5383/CHE/2013

(51) Int. Cl. C07H 23/00

(52) U.S. Cl. CPC C07H 23/00
USPC 536/17.1

(57) ABSTRACT

The present invention is directed to processes for preparation of iron sucrose complex and purification of the obtained iron sucrose through diafiltration.

### PRIORITY DOCUMENT

This patent application claims priority to Indian Provisional Patent Application number 5383/CHE/2013 filed on Nov. 21, 2013, the contents of which are incorporated by reference herein.

### INTRODUCTION

Aspects of the present application relates to process for the preparation and purification of Iron sucrose.

Iron Sucrose is a complex of polynuclear iron (III) hydroxide in sucrose having a molecular weight of approximately 34,000 to 60,000 daltons and a proposed structural formula:

[Na₂Fe₅O₈(OH).3(H₂O)]ₙ.m(C₁₂H₂₂O₁₁)

Where: n is the degree of iron polymerization and m is the number of sucrose molecules associated with the iron (III)-hydroxide. Iron Sucrose is an iron replacement product indicated for the treatment of iron deficiency anemia in patients with chronic kidney disease (CKD).

There are various prior art references, which disclose methods for preparation of complexes of Iron and Carbohydrates.

IN187116 discloses a method for preparation of Saccharated iron oxide in powder form having pH around 7 and water solubility suitable to use in tablets as well as for syrup preparations, wherein reaction of aqueous Ferric salt solution with aqueous solution of an sodium carbonate base to give Ferric hydroxide which on further treatment with sucrose at pH 6.5 to 7.5 gives Saccharated iron oxide complex.

U.S. Pat. No. 7,964,568 discloses a method for preparation of a high molecular weight Iron saccharidic complex wherein ferric hyroxide produced in situ from a ferric salt solution in presence of a base is contacted with sugars or sugar derivatives.

US2008/0167266A1 describes a method for the preparation of Iron sucrose complex wherein reaction of an aqueous solution of iron salt is mixed with sucrose and inorganic base selected from the group of alkali metal carbonate, alkali metal hydrogen carbonate at a temperature of −10° C. to 40° C. and a pH in the range of 3<pH<12 and pH of the reaction mixture is then raised to a value in the range of 10<pH<12 with an inorganic base selected from the group of alkali metal hydroxide, alkali metal carbonate and ammonium hydroxide and the reaction mixture is heated until the iron sucrose has completely formed, and the iron sucrose complex formed is then precipitated by mixing with a suitable water-miscible solvent.

IN1215-MUM-2007 describes a method for the preparation of Iron sucrose complex wherein reaction of ferric chloride with sodium hydroxide to get ferric hydroxide which on further reacting with sucrose in an aqueous reaction, at a selected molar ratio of sucrose to ferric hydroxide, for a selected time interval, at a selected temperature and pH and isolating the iron sucrose complex from the aqueous reaction mixture.

Further, IN1215-MUM-2007 describes a process for the preparation of iron sucrose complex wherein the reaction of ferric chloride and base in presence of water injection at selected ratios of water to ferric salt and base at selected temperature and time to get ferric hydroxide which on further treatment with sucrose in presence of hydrochloric acid and sodium hydroxide at selected temperature, pH and time followed by complexation with sucrose and base at specified conditions. Optionally iron sucrose complex isolated by organic solvents.

WO2009/078037A2 describes a method for the preparation of Iron sucrose complex wherein reaction of aqueous solution of ferric salt with a base to obtain ferric hydroxide at specified conditions which on further treatment with sucrose and inorganic base at specified reaction conditions and followed by autoclaving the reaction mixture at selected time and temperature and isolation of iron sucrose complex by organic solvents.

KR1017432B1 describes a method for the preparation of Iron sucrose complex wherein reaction of aqueous solution of ferric chloride with a base to obtain ferric oxy hydroxide which on further treatment with xylitol and sodium hydroxide to iron sucrose complex, which on further crystallization by ethanol.

U.S. Pat. No. 8,030,480 describes a method for the preparation of Iron sucrose complex wherein reaction of ferric salt solution with aqueous solution of an inorganic base at specified pH to give ferric oxy hydroxide, which on further treatment with sucrose solution and base at a selected temperature and pH to give iron sucrose complex.

US2008/0167266A1 describes a method for the purification of iron oxyhydroxide where it is purified through decantation, ion exchange, filtration or ultrafiltration.

The prior art processes suffer from one or more drawbacks such as inconsistency, less yield, not providing the desired physicochemical properties, not meeting the required quality, which does not result an industrially feasible process.

Therefore, there is a need to provide a simple, cost effective, industrially feasible process for the preparation of iron sucrose complex.

### SUMMARY

In one aspect, the application provides a process for preparation of iron sucrose complex having molecular weight of 34,000 to 60,000 daltons comprising:

(a) Preparing iron sucrose crude by the reaction of ferric oxyhydroxide, sucrose, base in an aqueous medium.

(b) Optionally subjecting the iron sucrose crude obtained in stage a) to diafiltration.

(c) Contacting the iron sucrose obtained in stage b) with additional sucrose to produce the iron-sucrose with desired molecular weight and particle size.

### DETAILED DESCRIPTION

In an aspect, the application provides a process for the preparation of iron-sucrose having molecular weight of 34,000 to 60,000 daltons comprising:

(a) Preparing iron sucrose crude by the reaction of ferric oxyhydroxide, sucrose, base in an aqueous medium.

(b) Optionally subjecting the iron sucrose crude obtained in stage a), to diafiltration

(c) Contacting the iron sucrose obtained in stage b) with additional sucrose to produce the iron-sucrose with desired molecular weight and particle size.

Stage a) involves the preparation of iron sucrose crude by the reaction of ferric oxyhydroxide, sucrose, base in an aqueous medium.

The crude iron sucrose of stage a) can be produced by the process comprising:

i. providing aqueous solution of iron salt;

ii. combining aqueous solution of sucrose with aqueous solution of iron salt as obtained in step i);

iii. adjusting pH of the reaction mixture to about 9 to 12 by using a base;

iv. heating the reaction mixture to a temperature at about 60° C. to about 120° C.;

v. optionally isolating the iron sucrose complex.

Step i) involves the preparation of an aqueous solution of iron salt. The aqueous solution of iron salt can be prepared by dissolving iron salt in water. In embodiments of step i), the iron salt that can be used for the preparation of aqueous iron salt solution include but are not limited to ferric chloride or any other suitable iron salt. The salts can be either hydrates or anhydrous in nature. The preferred iron salt is ferric chloride and more preferably ferric chloride hexahydrate.

In embodiments of step i) the aqueous solution of iron salt can be prepared by suspending the suitable iron salt in water. In embodiments, aqueous solution of iron salt can be prepared at any suitable temperatures, such as from about room temperature to about the boiling point of the water. Mixing may be used to reduce the time required for the dissolution process. In embodiments, aqueous solution of iron salt may be filtered to make it clear, free of undissolved particles. In embodiments, the obtained aqueous solution may be optionally treated with a decolorizing agent or an adsorbent material, such as carbon and/or hydrose, to remove colored components, etc., before filtration.

The ratio of iron salt and water is one of the parameter which plays a crucial role in determining the characteristics of final product, iron sucrose. In embodiment of step i) the ratio of iron salt to water can be from 1:2 to 1:10.

Step ii) involves the combining aqueous solution of sucrose to the aqueous solution of iron salt as obtained in step i).

In embodiment of step ii), the aqueous solution of sucrose is added to the aqueous solution of iron salt as obtained in step i). In embodiment of step ii), the aqueous solution of iron salt as obtained in step i) can be added to the aqueous solution of sucrose. In embodiments of step ii) solid sucrose in place of aqueous sucrose solution can also be added to the aqueous solution of iron salt. In embodiments of step ii) aqueous solution of sucrose or solid sucrose may be added to the aqueous solution of iron salt in one lot or in multiple lots.

In embodiments, aqueous solution of sucrose can be prepared by dissolving sucrose in water. In embodiments, aqueous solution of sucrose can be prepared at any suitable temperatures, such as from about room temperature to about the boiling point of the water. Mixing may be used to reduce the time required for the dissolution process. In embodiments, aqueous solution of sucrose may be filtered to make it clear, free of undissolved particles. In embodiments, the obtained aqueous solution of sucrose may be optionally treated with a decolorizing agent or an adsorbent material, such as carbon and/or hydrose, to remove colored components, etc., before filtration.

In embodiment of step ii), the aqueous solution of sucrose can be added to aqueous solution of iron salt as obtained in step i) or the aqueous solution of iron salt as obtained in step i) can be added to the aqueous solution of sucrose at a temperature at about 10 to 50° C., preferably at a temperature at about 20 to 40° C. In embodiments, the reaction mixture obtained in step ii) can be maintained for 10 minutes to 5 hours or longer.

Alternatively, the iron oxyhydroxyde can be prepared in situ or it can be prepared independently before the addition of sucrose.

Step iii) involves the pH adjustment of the reaction mixture as obtained in step ii) by using a suitable base.

In embodiment of step iii), the base which may be used for pH adjustment includes but are not limited to alkali metal hydroxide or alkali metal hydrogen carbonate or alkaline metal carbonate or ammonium hydroxide or the like or any other suitable base. Preferably the base can be sodium hydroxide. In embodiments, the aqueous solution of sodium hydroxide can be added to the reaction mixture. In embodiments, the solid sodium hydroxide can also be added to adjust the pH. In embodiment of step iii), pH of the reaction mass can be adjusted to about 9 to 12. In embodiments of step iii), the aqueous solution of sodium hydroxide or solid sodium hydroxide can be added to reaction mass in one lot or multiple lots.

In embodiments of step iii), the aqueous solution of sodium hydroxide or solid sodium hydroxide can be added to reaction mass in about 10 minutes to 5 hours or longer at temperature about 10° C. to 50° C.

Step iv) involves heating of the reaction mixture as obtained in step iii) to a temperature at about 60° C. to about 120° C. In embodiments of step iv), reaction mixture as obtained in step iii) can be heated to a temperature at about 60° C. to about 120° C. In embodiments of step iv), preferably reaction mixture can be heated to a temperature at about 80° C. to about 100° C. In embodiments of step iv), the reaction mixture can be maintained for 10 minutes to 5 hours or longer at the temperature as mentioned above. In embodiments, the rate of stirring i.e. RPM (rotations per minute) during the maintenance also plays an important role for getting the desired physicochemical properties of the product. In embodiments, the stirring can be done at about 100 RPM to about 450 RPM, preferably at about 200 RPM to about 300 RPM.

In an embodiment, when the ferric salt, sucrose and base are added initially in any order, the ferric oxyhydroxide formed in situ will react with sucrose to produce crude iron sucrose complex.

In embodiments, after completion of the reaction, the reaction mass was cooled to 25-35° C. In embodiments, the obtained aqueous solution containing the product can be used for the next step for further purification or optionally combined with an organic solvent to precipitate the product. In embodiments, the organic solvent can be water miscible solvent. Preferably the water miscible organic solvent can be ethanol. In embodiments, the obtained Iron sucrose complex can be isolated in solid form or liquid form.

The iron oxyhydroxide monohydrate (FeO(OH).H₂O) might otherwise be described as iron(III) hydroxide (Fe(OH)₃), and is also known as hydrated iron oxide or yellow iron oxide. Ferric oxyhydroxide is also called as Iron (III) hydroxide, Ferric hydroxide, Iron oxide yellow 313, Iron oxide yellow HD718, Iron(III)oxide hydrated etc.

In embodiments, the obtained Iron sucrose complex can be isolated by using any techniques, such as decantation, filtration by gravity or suction, centrifugation, or the solvent can be evaporated from the mass to obtain the desired product, and optionally the solid can be washed with a solvent, such as the solvent used for the crystallization to reduce the amount of entrained impurities in the product. In embodiments, the obtained Iron sucrose complex can be isolated by filtration.

In embodiments, if the iron sucrose complex isolated, it can be dried at suitable temperatures such as room temperature to about 80° C. under atmospheric or reduced pressures, for about 10 minutes to about 50 hours, or longer, using any types of drying equipment, such as a tray dryer, vacuum oven, air oven, fluidized bed dryer, spin flash dryer, flash dryer, or the like.

The crude iron sucrose complex can be produced as per the methods described herein or as per the methods known in the literature.

The crude iron sucrose complex obtained by the methods described herein or produced as per the methods known in the literature can be used for the diafiltration step ind subsequent sucrose addition for getting the desired quality product. The crude iron sucrose complex if isolated as a solid can be dissolved in water again before performing the diafiltration and additional sucrose addition steps.

Stage b) involves the purification of the solution containing the product i.e., iron sucrose complex by diafiltration.

The term diafiltration refers to a technique that uses filtration membranes to completely remove, replace, or lower the concentration of salts, solvents or low molecular weight species from solutions containing high molecular weight species by selectively utilizing permeable (porous) membrane filters to separate the components of solutions and suspensions based on their molecular size. The process involves filtering the feed solution through a membrane under pressure. The solution which is subjected to filtration is called feed solution. The solution which is filtered through membrane is called as permeate and the solution which is retained is called as retentate. The step of diafiltration will lead to the reduction of low molecular weight compounds, unbound sucrose, loosely bound sucrose, unwanted byproducts etc.

In embodiments of stage b), the solution containing the product i.e., iron sucrose complex as obtained in step iv) or iron sucrose crude obtained by any of the known processes is purified by using diafiltration technique. In embodiments, solution of iron sucrose complex is subjected to filtration through ultrafiltration membranes. The aqueous solution containing iron sucrose was allowed to concentrate though diafiltration and a basic buffer solution is added to compensate the loss of volume. One of such cycle is called one diafiltration. This diafiltration cycle can be repeated till the desired quality of the product is obtained. Solid sucrose or aqueous solution of sucrose can be optionally added to the above obtained solution during the diafiltration.

In the process, the reaction mixture of step iv) is subjected to filtration through a membrane. The membrane can be selected from the group which is stable at high pH like polyether sulfone, polysulphone, PVDF, Ceramic membranes either in spiral, tubular or hollow fiber. The pore size based on cut-off of the membrane can be about 1 kilodalton to 30 kilodalton (kD). The pore size of the membrane is preferably 5 kilodalton to 15 kilodalton. The flow rate of the system can be maintained at 80-300 meters per hour (mPH). If desired, the retentate can be further subjected to diafiltration process till desired characteristics of the product are achieved.

Stage c) involves contacting the iron sucrose obtained in stage b) with additional sucrose to produce the iron-sucrose with desired molecular weight and particle size.

In embodiment of stage c), the addition of sucrose can be carried out in a single or multiple lots followed by the addition of a base for making the desired pH.

In embodiment of stage c), sucrose can be added as a solid or in the form of an aqueous solution. Sucrose may be added in one lot or in multiple lots.

In embodiment of stage c), the base which may be used for pH adjustment includes but are not limited to alkali metal hydroxide or alkali metal hydrogen carbonate or alkaline metal carbonate or ammonium hydroxide or the like or any other suitable base. Preferably the base can be sodium hydroxide. In embodiments, the aqueous solution of sodium hydroxide can be added to the reaction mixture. In embodiments, the solid sodium hydroxide can also be added to adjust the pH. In embodiment of stage c), pH of the reaction mass can be adjusted to about 9 to 12. In embodiments of stage c), the aqueous solution of sodium hydroxide or solid sodium hydroxide can be added to reaction mass in one lot or multiple lots for obtained the desired pH.

In embodiments, aqueous solution of sucrose can be prepared by dissolving sucrose in water. In embodiments, aqueous solution of sucrose can be prepared at any suitable temperatures, such as from about room temperature to about the boiling point of the water. Mixing may be used to reduce the time required for the dissolution process. In embodiments, aqueous solution of sucrose may be filtered to make it clear, free of undissolved particles.

In embodiment of stage c), during or after the addition of sucrose optionally any other sugar or sugar derivative, which is different from sucrose, can be added for developing the desired molecular weight and particle size.

In an embodiment of stage c), involves heating of the reaction mixture to a temperature at about 60° C. to about 120° C. after the addition of sucrose and base. In embodiments of stage c), the reaction mixture to be heated to a temperature at about 60° C. to about 120° C. In embodiments of stage c), preferably reaction mixture can be heated to a temperature at about 80° C. to about 110° C. In embodiments of stage c), the reaction mixture can be maintained for 10 minutes to 5 hours or longer at the temperature as mentioned above. In embodiments, the rate of stirring i.e. RPM (rotations per minute) during the maintenance also plays an important role for getting the desired physicochemical properties of the product. In embodiments, the stirring can be done at about 100 RPM to about 450 RPM, preferably at about 200 RPM to about 300 RPM.

Optionally, water is added to the reaction mass obtained in stage c) to achieve the desired concentration of iron sucrose complex. The aqueous solution of iron sucrose complex can be used as such in the formulations.

The isolation and drying of the product can be followed by using the procedures as described in the first aspect of the application for the isolating purpose, if intended.

All the steps and parameters play an important role in obtaining the iron sucrose with desired quality that meet the requirements.

The obtained iron sucrose complex meets the regulatory requirements provided by USFDA and the product is also in compliance with US pharmacopeia.

Desired quality of the product is the iron sucrose complex which meets the regulatory requirements provided by USFDA and the product is also in compliance with US pharmacopeia. As per the USP, the molecular weight (Mw) of iron sucrose complex is ranging between 34,000 to 60,000 daltons and USP also indicates various other requirements.

The obtained iron sucrose complex is useful for preparing pharmaceutical compositions containing a therapeutically effective amount of iron sucrose complex, together with or without pharmaceutically acceptable excipients.

The pharmaceutical compositions comprising iron sucrose complex of the invention together with or without pharmaceutically acceptable excipients may be formulated as: solid oral dosage forms, such as, but not limited to: powders, granules, pellets, tablets, and capsules; liquid oral dosage forms such as but not limited to syrups, suspensions, dispersions, and emulsions; and injectable preparations such as, but not limited to, solutions, dispersions, and freeze-dried compositions. Formulations may be in the form of immediate release, delayed release or modified release. Further, immediate release compositions may be conventional, dispersible, chewable, mouth dissolving, or flash melt preparations, and modified release compositions may comprise hydrophilic or hydrophobic, or combinations of hydrophilic and hydrophobic, release rate-controlling substances to form matrix or reservoir systems, or combinations of matrix and reservoir systems. The compositions may be prepared using any one or more of techniques such as direct blending, dry granulation, wet granulation, and extrusion and spheronization. Compositions may be presented as uncoated, film coated, sugar coated powder coated, enteric coated, or modified release coated.

Certain specific aspects and embodiments of the present application will be explained in greater detail with reference to the following example, which are provided only for purposes of illustration and should not be construed as limiting the scope of the application in any manner. Variations of the described procedures, as will be apparent to those skilled in the art, are intended to be within the scope of the present application.

1. A process for the preparation of iron-sucrose having molecular weight of 34,000 to 60,000 daltons comprising:
(a) preparing iron sucrose crude by the reaction of ferric oxyhydroxide, sucrose, base in an aqueous medium;
(b) optionally subjecting the iron sucrose crude obtained in stage (a), to diafiltration; and
(c) contacting the iron sucrose obtained in stage (b) with additional sucrose to produce the iron-sucrose with desired molecular weight and particle size.

2. The process of claim 1, wherein in stage (a), the ferric oxyhydroxide is prepared in situ by the reaction of ferric salt with a base.

3. The process of claim 2, wherein the ferric salt is ferric chloride or its hydrate.

4. The process of claim 1, wherein in stage (a), the ferric salt, sucrose and base are added initially in any order, the ferric oxyhydroxide formed in situ will react with sucrose to produce crude iron sucrose complex.

5. The process of claim 1, wherein the iron sucrose complex formation takes place at a temperature of about 60° C. to about 120° C.

6. The process according to claim 1, wherein the iron sucrose complex formation takes place at a pH of 6 to 12.

7. The process of claim 1, wherein in stage (b), the diafiltration is carried out repeatedly till the desired quality is achieved.

8. The process of claim 1, wherein stage (c), additional sucrose and base are added to achieve the desired molecular weight.

9. The process of claim 8, wherein in stage (c), the reaction mass is heated to about 60° C. to about 120° C. after the addition of sucrose and base to get the desired molecular weight.
